(19) United States
(12) Patent Application Publication
(10) Pub. No.: US 2011/0229939 A1
(43) Pub. Date: Sep. 22, 2011

(54) METHOD FOR AMPLIFYING DOUBLE STRANDED TARGET SEQUENCE IN DOUBLE STRANDED DNA

(75) Inventors: HIDENOBU YAKU, OSAKA (JP); MIHO HAYASHI, EHIME (JP)

(73) Assignee: PANASONIC CORPORATION, OSAKA (JP)

(21) Appl. No.: 12/955,594

(22) Filed: Nov. 29, 2010

Related U.S. Application Data
(63) Continuation of application No. PCT/JP2010/001886, filed on Mar. 16, 2010.

(51) Int. Cl. C12P 19/34

(52) U.S. Cl. 435/91.2

(57) ABSTRACT

The present invention relates to a nested PCR with high specificity. The present invention provides a method for amplifying a target sequence (1), and the method demonstrates high efficiency of amplification of the single stranded target sequence and a significant effect on inhibiting nonspecific amplifications. In one embodiment, at the second stage of a nested PCR, an outer forward block nucleic acid (4ofb) which is complementary to an outer forward primer (4of) and which is unable to be an origin of a DNA extension reaction by the DNA polymerase is added.

### CROSS-REFERENCE TO RELATED APPLICATION

This is a continuation of PCT International Application PCT/JP2010/001886 filed on Mar. 16, 2010, the disclosure of which application is hereby incorporated by reference in its entirety.

### TECHNICAL FIELD

The present invention relates to a nested PCR with high specificity.

### BACKGROUND ART

A Nested Polymerase Chain Reaction (hereinafter, referred to as “nested PCR”) shown in FIG. 1 is a representative method for amplifying a double stranded target sequence 1 that is included in a double stranded DNA consisting of a first single stranded DNA 6 and a second single stranded DNA 7.

The nested PCR method will be described briefly in the following with reference to FIG. 1.

The first single stranded DNA 6 consists of a 3′ end—a first unamplified sequence 6a—a second unamplified sequence 6b—a single stranded target sequence 1a—a third unamplified sequence 6c—a fourth unamplified sequence 6d—a 5′ end. The second single stranded DNA 7 consists of a 5′ end—a fifth unamplified sequence 7a—a sixth unamplified sequence 7b—a complementary single stranded target sequence 1b—a seventh unamplified sequence 7c—an eighth unamplified sequence 7d—a 3′ end.

The fifth unamplified sequence 7a, the sixth unamplified sequence 7b, the complementary single stranded target sequence 1b, the seventh unamplified sequence 7c, and the eighth unamplified sequence 7d are complementary to the first unamplified sequence 6a, the second unamplified sequence 6b, the single stranded target sequence 1a, the third unamplified sequence 6c, and the fourth unamplified sequence 6d, respectively.

The double stranded target sequence consists of the single stranded target sequence 1a and the complementary single stranded target sequence 1b.

To start with, DNA polymerase, deoxynucleoside triphosphate, the double stranded DNA (6•7), an outer forward primer (4of), and an outer reverse primer (5or) are mixed to prepare a first mixture.

The outer forward primer (4of) consists of a nucleic acid of 5 to 40 bases, and is complementary to a 3′ end sequence portion included in the second unamplified sequence 6b. The outer reverse primer (5or) consists of a nucleic acid of 5 to 40 bases, and is complementary to a 3′ end sequence portion included in the seventh unamplified sequence 7c. Therefore, the outer forward primer (4of) and the outer reverse primer (5or) bind to a 3′ end sequence portion included in the second unamplified sequence 6b and a 3′ end sequence portion included in the seventh unamplified sequence 7c, respectively.

Next, the first mixture is heated for a duration of 1 second to 100 seconds at 94° C. to 100° C. Then, the mixture is cooled for 1 second to 100 seconds at 50° C. to 70° C. Furthermore, the mixture is heated for 1 second to 600 seconds at 70° C. to 80° C. These steps are repeated to amplify an intermediate double stranded DNA.

The intermediate double stranded DNA is a double stranded DNA that consists of an intermediate single stranded target sequence 6m and a complementary single stranded intermediate target sequence 7m. The intermediate single stranded target sequence and the complementary single stranded intermediate target sequence consist of a 3′ end—the second unamplified sequence 6b—the single stranded target sequence 1a—the third unamplified sequence 6c—a 5′ end and a 5′ end—the sixth unamplified sequence 7b—the complementary single stranded target sequence 1b—the seventh unamplified sequence 7c—a 3′ end, respectively.

The steps up to this point are referred to as “the first stage of PCR”.

Next, the second stage of PCR will be conducted.

The amplified intermediate double stranded DNA, DNA polymerase, deoxynucleoside triphosphate, an inner forward primer (4if), and an inner reverse primer (5ir) are mixed to prepare a second mixture.

The inner forward primer (4if) consists of a nucleic acid of 5 to 40 bases, and is complementary to a 3′ end sequence portion included in the single stranded target sequence (1a). The inner reverse primer (5ir) consists of a nucleic acid of 5 to 40 bases, and is complementary to a 3′ end sequence portion included in the complementary single stranded target sequence 1b. Therefore, the inner forward primer (4if) and the inner reverse primer (5ir) bind to a 3′ end sequence portion included in the single stranded target sequence 1a and a 3′ end sequence portion included in the complementary single stranded target sequence 1b, respectively.

Lastly, the second mixture is heated for a duration of 1 second to 100 seconds at 94° C. to 100° C. Then, the mixture is cooled for 1 second to 100 seconds at 50° C. to 70° C. Furthermore, the mixture is heated for 1 second to 600 seconds at 70° C. to 80° C. These steps are repeated to amplify the double stranded target sequence.

Patent literature 1 and non-patent literatures 1 to 3 can be relevant to the present invention.

### CITATION LIST

### Patent Literature

[PTL 1] (PCT) International Publication WO96/17932

### Non Patent Literature

[NPL 1] Genome Research, 4, 376-379 (1995)
[NPL 2] Genome Research, 2, 60-65 (1992)
[NPL 3] Phytopathology, 86, 493-497 (1996)

### SUMMARY OF THE INVENTION

### Problems to be Solved by the Invention

After the first stage of PCR, it is necessary to remove the outer primers 4of•5or before conducting the second stage of PCR. This is because, if the second mixture contains the outer primers 4of•5or, DNA extension reactions occur also from the outer primers at the second stage of PCR as shown in FIG. 2.

Thus, as shown in FIG. 2, not only the desired target double stranded DNA, but an undesired unnecessary amplification product is also obtained after the second stage of PCR. The undesired unnecessary amplification product can cause a significant decrease in efficiency of amplification of the target double stranded DNA, difficulty of DNA analysis by electrophoresis, and an error in genetic diagnosis.

An objective of the present invention is to provide a method for efficiently amplifying a target sequence (1) by utilizing a nested PCR, and which method inhibits production of the undesired unnecessary amplification product.

### Solution to the Problems

In order to solve the above-described problem, the present invention is provided in modes illustrated as follows.

(Item 1) A method for amplifying a double stranded target sequence (1) in a double stranded DNA consisting of a first single stranded DNA (6) and a second single stranded DNA (7), wherein

the double stranded target sequence (1) consists of a single stranded target sequence (1a) and a complementary single stranded target sequence (1b),

the first single stranded DNA (6) consists of a 3′ end—a first unamplified sequence (6a)—a second unamplified sequence (6b)—the single stranded target sequence (1a)—a third unamplified sequence (6c)—a fourth unamplified sequence (6d)—a 5′ end,

the second single stranded DNA (7) consists of a 5′ end—a fifth unamplified sequence (7a)—a sixth unamplified sequence (7b)—the complementary single stranded target sequence (1b)—a seventh unamplified sequence (7c)—an eighth unamplified sequence (7d)—a 3′ end, and

the complementary single stranded target sequence (1b), the fifth unamplified sequence (7a), the sixth unamplified sequence (7b), seventh unamplified sequence (7c), and the eighth unamplified sequence (7d) are complementary to the single stranded target sequence (1a), the first unamplified sequence (6a), the second unamplified sequence (6b), the third unamplified sequence (6c), and the fourth unamplified sequence (6d), respectively, and

the method comprising the following step (A) and step (B):

the step (A) of mixing DNA polymerase, deoxynucleoside triphosphate, the double stranded DNA (6•7), an outer forward primer (4of), and an outer reverse primer (5or) to amplify an intermediate double stranded DNA by utilizing a polymerase chain reaction, wherein

the intermediate double stranded DNA consists of an intermediate target sequence and a complementary intermediate target sequence,

the intermediate target sequence consists of a 3′ end—the second unamplified sequence (6b)—the single stranded target sequence (1a)—the third unamplified sequence (6c)—a 5′ end,

the complementary intermediate target sequence consists of a 5′ end—the sixth unamplified sequence (7b)—the complementary single stranded target sequence (1b)—the seventh unamplified sequence (7c)—a 3′ end,

the outer forward primer (4of) is complementary to a 3′ end sequence portion included in the second unamplified sequence (6b), and

the outer reverse primer (5or) is complementary to a 3′ end sequence portion included in the seventh unamplified sequence; and

the step (B) of mixing DNA polymerase, deoxynucleoside triphosphate, the intermediate double stranded DNA, an inner forward primer (4if), an inner reverse primer (5ir), and an outer forward block nucleic acid (4ofb) to amplify specifically the target sequence (1) by utilizing a polymerase chain reaction, wherein

the inner forward primer (4if) is complementary to a 3′ end sequence portion included in the single stranded target sequence (1a),

the inner reverse primer (5ir) is complementary to a 3′ end sequence portion included in the complementary single stranded target sequence (1b), and

the outer forward block nucleic acid (4ofb) is complementary to the outer forward primer (4of) and unable to be an origin of a DNA extension reaction by the DNA polymerase.

(Item 2) The method according to item 1, wherein

an outer reverse block nucleic acid (5orb) is additionally mixed at the step (B), and

the outer reverse block nucleic acid (5orb) is complementary to the outer reverse primer (5or) and is unable to be an origin of a DNA extension reaction by the DNA polymerase.

(Item 3) The method according to item 1, wherein the outer forward block nucleic acid (4ofb) consists of a DNA in which an OH group at carbon number 3 of a sugar included in a nucleotide located at a 3′ end is substituted or modified by a hydrogen, phosphate group, amino group, biotin group, thiol group, or a derivative thereof.
(Item 4) The method according to item 1, wherein the outer forward block nucleic acid (4ofb) consists of a Locked Nucleic Acid in which an OH group at carbon number 3 of a sugar included in a nucleotide located at a 3′ end is substituted or modified by a hydrogen, phosphate group, amino group, biotin group, thiol group, or a derivative thereof.
(Item 5) The method according to item 1, wherein the outer forward block nucleic acid (4ofb) consists of a Peptide Nucleic Acid.
(Item 6) The method according to item 2, wherein the outer reverse block nucleic acid (5orb) consists of a DNA in which an OH group at carbon number 3 of a sugar included in a nucleotide located at a 3′ end is substituted or modified by a hydrogen, phosphate group, amino group, biotin group, thiol group, or a derivative thereof.
(Item 7) The method according to item 2, wherein the outer reverse block nucleic acid (5orb) consists of a Locked Nucleic Acid in which an OH group at carbon number 3 of a sugar included in a nucleotide located at a 3′ end is substituted or modified by a hydrogen, phosphate group, amino group, biotin group, thiol group, or a derivative thereof.
(Item 8) The method according to item 2, wherein the outer reverse block nucleic acid (5orb) consists of a Peptide Nucleic Acid.
(Item 9) A method for amplifying a double stranded target sequence (1) in a double stranded DNA consisting of a first single stranded DNA (6) and a second single stranded DNA (7), wherein

the double stranded target sequence (1) consists of a single stranded target sequence (1a) and a complementary single stranded target sequence (1b),

the first single stranded DNA (6) consists of a 3′ end—a first unamplified sequence (6a)—a second unamplified sequence (6b)—the single stranded target sequence (1a)—a third unamplified sequence (6c)—a fourth unamplified sequence (6d)—a 5′ end,

the second single stranded DNA (7) consists of a 5′ end—a fifth unamplified sequence (7a)—a sixth unamplified sequence (7b)—the complementary single stranded target sequence (1b)—a seventh unamplified sequence (7c)—an eighth unamplified sequence (7d)—a 3′ end, and

the complementary single stranded target sequence (1b), the fifth unamplified sequence (7a), the sixth unamplified sequence (7b), the seventh unamplified sequence (7c), and the eighth unamplified sequence (7d) are complementary to the single stranded target sequence (1a), the first unamplified sequence (6a), the second unamplified sequence (6b), the third unamplified sequence (6c), and the fourth unamplified sequence (6d), respectively, and

the method comprising the following step (A) and step (B):

the step (A) of mixing DNA polymerase, deoxynucleoside triphosphate, the double stranded DNA (6•7), an outer forward primer (4of), and an inner reverse primer (5ir) to amplify an intermediate double stranded DNA by utilizing a polymerase chain reaction, wherein

the intermediate double stranded DNA consists of an intermediate target sequence and a complementary intermediate target sequence, the intermediate target sequence consists of a 3′ end—the second unamplified sequence (6b)—the single stranded target sequence (1a)—a 5′ end,

the complementary intermediate target sequence consists of a 5′ end—the sixth unamplified sequence (7b)—the complementary single stranded target sequence (1b)—a 3′ end,

the outer forward primer (4of) is complementary to a 3′ end sequence portion included in the second unamplified sequence (6b), and

the inner reverse primer (5ir) is complementary to a 3′ end sequence portion included in the complementary single stranded target sequence (1b); and

the step (B) of mixing DNA polymerase, deoxynucleoside triphosphate, the intermediate double stranded DNA, an inner forward primer (4if), and an outer forward block nucleic acid (4ofb) to amplify specifically the target sequence (1) by utilizing a polymerase chain reaction, wherein

the inner forward primer (4if) is complementary to a 3′ end sequence portion included in the single stranded target sequence (1a),

the outer forward block nucleic acid (4ofb) is complementary to the outer forward primer (4of) and unable to be an origin of a DNA extension reaction by the DNA polymerase.

(Item 10) The method according to item 9, wherein the outer forward block nucleic acid (4ofb) consists of a DNA in which an OH group at carbon number 3 of a sugar included in a nucleotide located at a 3′ end is substituted or modified by a hydrogen, phosphate group, amino group, biotin group, thiol group, or a derivative thereof.
(Item 11) The method according to item 9, wherein the outer forward block nucleic acid (4ofb) consists of a Locked Nucleic Acid in which an OH group at carbon number 3 of a sugar included in a nucleotide located at a 3′ end is substituted or modified by a hydrogen, phosphate group, amino group, biotin group, thiol group, or a derivative thereof.
(Item 12) The method according to item 9, wherein the outer forward block nucleic acid (4ofb) consists of a Peptide Nucleic Acid.

### Advantageous Effects of the Invention

The present invention provides a method which is for amplifying a target sequence and which is capable of improving amplification efficiency and significantly inhibiting nonspecific amplifications.

### BRIEF DESCRIPTION OF THE DRAWINGS

FIG. 1 is a figure showing a conventional nested PCR.

FIG. 2 is a figure showing a problematic point of the conventional nested PCR, where DNA extension reactions occur from outer primers at the second stage of PCR.

FIG. 3 is a figure showing a nested PCR according to the present embodiment 1.

FIG. 4 is a figure showing a problematic point in the case where block primers are used instead of block nucleic acids of the present embodiment 1.

FIG. 5 is a figure showing a nested PCR according to the present embodiment 2.

FIG. 6 is a figure showing electrophoresis results from profile 1A in Comparative Example 1a (FIG. 6 (A)) and Example 1 (FIG. 6 (B)).

FIG. 7 is a figure showing electrophoresis results from profile 1B in Comparative Example 1a (FIG. 7 (A)) and Example 1 (FIG. 7 (B)).

FIG. 8 is a figure showing electrophoresis results from profile 1C in Comparative Example 1a (FIG. 8 (A)) and Example 1 (FIG. 8 (B)).

FIG. 9 is a figure in which nonspecific amplifications in Example 1 and Comparative Example 1a are compared quantitatively.

FIG. 10 is a figure showing an electrophoresis result from profile 1A in Comparative Example 1b (FIG. 8 (A)).

FIG. 11 is a figure showing electrophoresis results from profile 2A in Example 2 and Comparative Example 2.

FIG. 12 is a figure showing electrophoresis results from profile 2B in Example 2 and Comparative Example 2.

FIG. 13 is a figure showing electrophoresis results from profile 2C in Example 2 and Comparative Example 2.

FIG. 14 is a figure in which nonspecific amplifications in Example 2 and Comparative Example 2 are compared quantitatively.

FIG. 15 is a figure showing electrophoresis results from profile 3A in Comparative Example 3a (FIG. 15 (A)) and Example 3 (FIG. 15 (B)).

FIG. 16 is a figure showing electrophoresis results from profile 3B in Comparative Example 3a (FIG. 16 (A)) and Example 3 (FIG. 16 (B)).

FIG. 17 is a figure showing an electrophoresis result from profile 3A in Comparative Example 3b.

### MODES FOR CARRYING OUT THE INVENTION

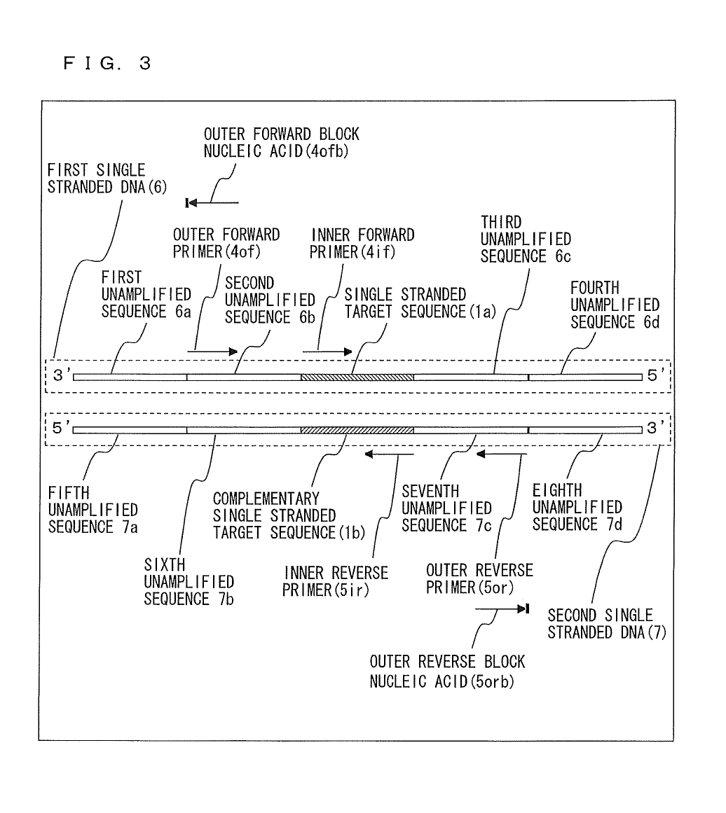

Embodiments of the present invention will be described in the following with reference to FIG. 3.

### Embodiment 1

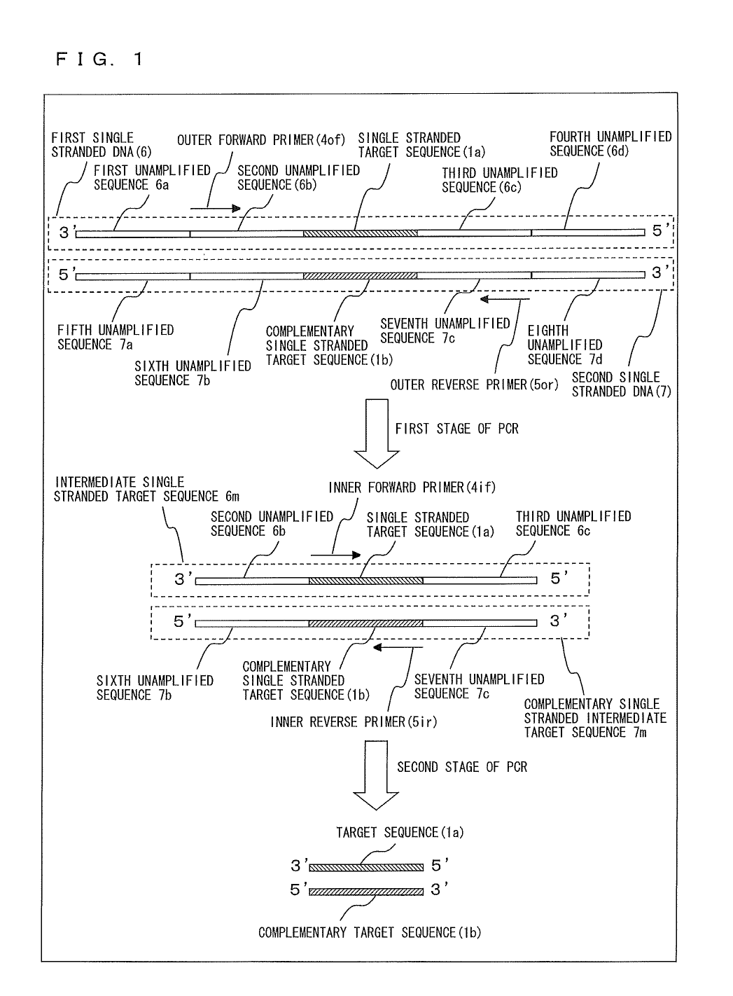

In the present embodiment, to start with, the first stage of PCR is conducted by using the outer primers as indicated in FIG. 1. In the first stage of PCR, a mixture does not contain inner primers.

As shown in FIG. 3, the present embodiment can be characterized by a usage of an outer forward block nucleic acid (4ofb) at the second stage of PCR. Preferably, both the outer forward block nucleic acid (4ofb) and an outer reverse block nucleic acid (5orb) are used.

The outer forward block nucleic acid (4ofb) and the outer reverse block nucleic acid (5orb) have sequences that are complementary to an outer forward primer (4of) and an outer reverse primer (5or), respectively. Furthermore, none of the block nucleic acids (4ofb•5orb) act as an origin for an extension reaction by DNA polymerase. Preferably, the block nucleic acids (4ofb•5orb) are synthetic oligonucleic acids.

Examples of the block nucleic acids (4ofb•5orb) are modified DNA, modified Locked Nucleic Acid (“LNA” hereinafter), and Peptide Nucleic Acid (“PNA” hereinafter).

A nucleic acid is a biological macromolecule in which multiple nucleotides, each of which comprising a sugar, a phosphate group, and a base, are linked via phosphodiester bonds. In a modified DNA and a modified LNA, an OH group at carbon number 3 of a sugar included in a nucleotide located at a 3′ end is substituted or modified by a hydrogen, phosphate group, amino group, biotin group, thiol group or a derivative thereof. An LNA is a nucleic acid analog developed artificially. A PNA does not require the above described modification. This is because, in a PNA, the sugar-phosphate diester backbone is substituted with a (2-aminoethyl)-glycine linkage.

Before initiating the second stage of PCR, an inner forward primer (4if) and an inner reverse primer (5ir) are added to the mixture. Furthermore, the outer forward block nucleic acid (4ofb) is added to the mixture. It is preferable to have the outer reverse block nucleic acid (5orb) also being added.

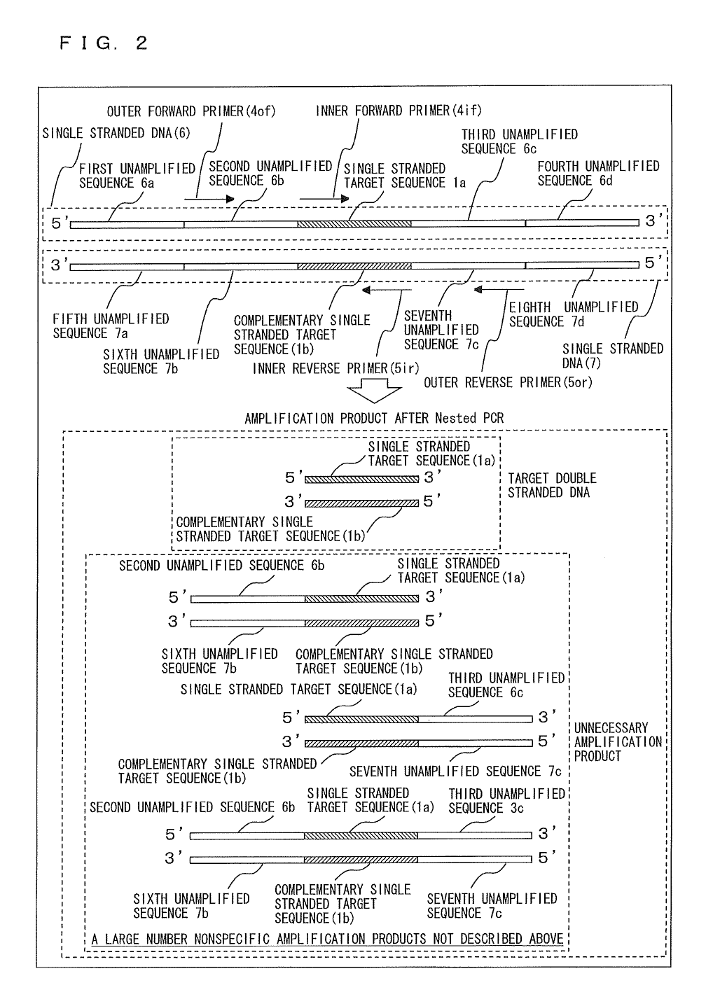

When the block nucleic acids (4ofb•5orb) are added, the outer forward block nucleic acid (4ofb) binds to the outer forward primer (4of) to form a double stranded DNA structure called a primer dimer. Similarly, the outer reverse block nucleic acid (5orb) binds to the outer reverse primer (5or) to form the double stranded DNA structure. The formation of these double stranded DNA structures reduces activities of the outer forward primer (4of) and the outer reverse primer (5or). Therefore, a DNA extension reaction from outer primers at the second stage of PCR, as shown in FIG. 2, is inhibited.

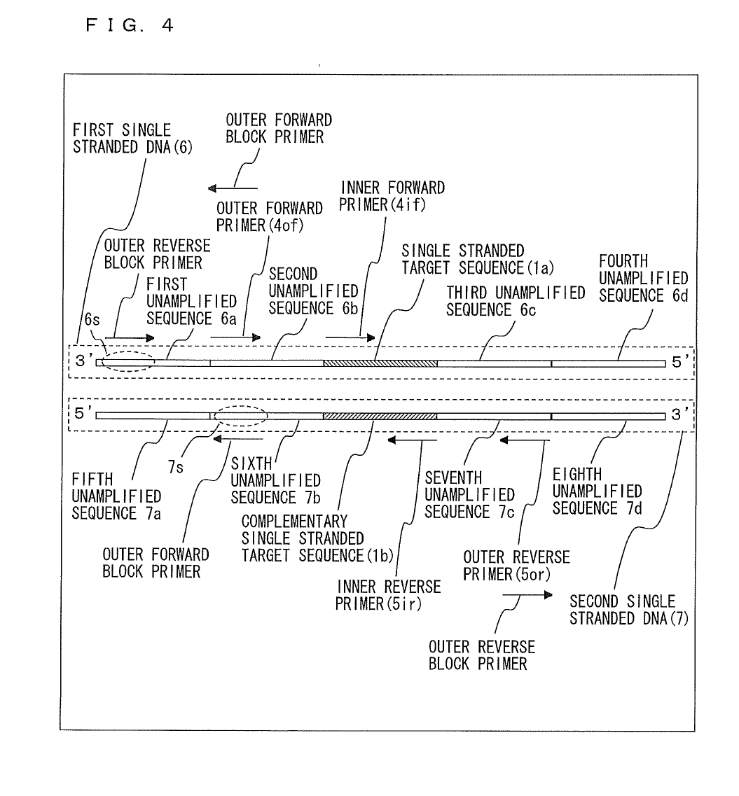

The outer forward block nucleic acid (4ofb) should not be merely a primer that is complementary to the outer forward primer (4of). Specifically, the outer forward block nucleic acid (4ofb) should not be an origin of a DNA extension reaction by the DNA polymerase. The reason for this will be described in the following with reference to FIG. 4.

As shown in FIG. 4, and similarly in FIG. 3, the outer forward block primer forms a double stranded DNA structure together with the outer forward primer (4of). However, when the second single stranded DNA 7 includes a sequence portion 7s that is identical or similar to a sequence complementary to the outer forward block primer, the outer forward block primer also binds to the sequence portion 7s. A DNA extension reaction is then initiated from the outer forward block primer. This will produce an unnecessary amplification product as shown in FIG. 2. Similarly, the outer reverse block nucleic acid (5orb) also should not be merely a primer that is complementary to the outer reverse primer (5or). This is because, when the first single stranded DNA 6 includes a sequence portion 6s that is identical or similar to a sequence complementary to the outer reverse block primer,

the sequence portion 6s binds to the outer reverse block primer and initiates a DNA extension reaction. When the first single stranded DNA 6 and the second single stranded DNA 7 include multiple similar sequences 6s and multiple similar sequences 7s, respectively, large quantities of undesired amplification products can be produced. For details, please refer to Example 1 along with Comparative Example 1b, and Example 3 along with Comparative Example 3b.

It is preferable to have the concentration of the outer forward block nucleic acid (4ofb) to be higher than the concentration of the outer forward primer (4of). More specifically, it is preferable to have the concentration of the outer forward block nucleic acid (4ofb) to be 5 times of that of the outer forward primer (4of), and it is more preferable to have the concentration to be 10 times of that of the outer forward primer (4of).

It is preferable to also have the concentration of the outer reverse block nucleic acid (5orb) to be higher than the concentration of the outer reverse primer (5or). More specifically, it is preferable to have the concentration of the outer reverse block nucleic acid (5orb) to be 5 times of that of the outer reverse primer (5or), and it is more preferable to have the concentration to be 10 times of that of the outer reverse primer (5or).

Similar to a common PCR, in the first stage of PCR and in the second stage of PCR, reagents such as, a component having a pH buffering effect, a salt such as MgCl₂, dithiothreitol, bovine serum albumin, and glycerol can also be mixed as necessary.

### Embodiment 2

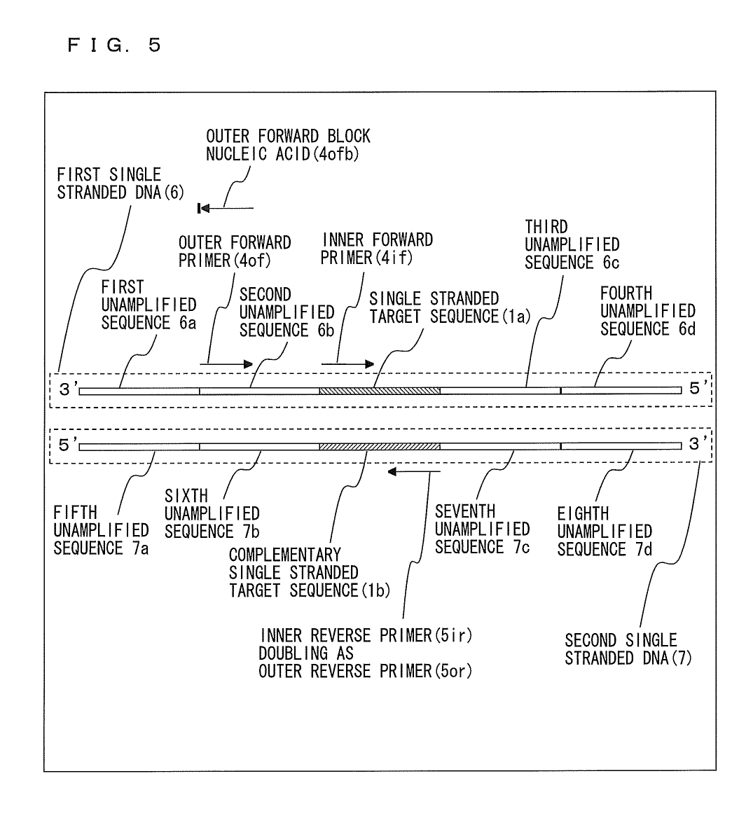

The present embodiment 2 will be described with reference to FIG. 5. The difference between the present embodiment 2 and embodiment 1 is that the inner reverse primer (5ir) doubles as the outer reverse primer (5or).

Also in the present embodiment 2, to start with, the first stage of PCR is conducted by using the outer primer as indicated in FIG. 1. Unlike embodiment 1, the mixture contains the inner reverse primer (5ir) in the first stage of PCR in embodiment 2. The first stage of PCR produces an intermediate single stranded target sequence consisting of a second unamplified sequence 6b—a single stranded target sequence 1a, and a complementary single stranded intermediate target sequence consisting of a sixth unamplified sequence 7b—a complementary single stranded target sequence 1b.

Before the second stage of PCR, the outer forward block nucleic acid (4ofb) is mixed. Unlike embodiment 1, the outer reverse block nucleic acid (5orb) is not mixed. The second stage of PCR amplifies the single stranded target sequence 1a and the complementary single stranded target sequence 1b, with the inner forward primer (4if) and the inner reverse primer (5ir).

### EXAMPLES

A template DNA used in the present Example and Comparative Example was prepared from a human blood sample by using an Automated DNA Extraction Device QIAcube (manufactured by QIAGEN Inc.).

All primers, outer forward block nucleic acids, and outer reverse block nucleic acids were purchased from Tsukuba Oligo Service Co., Ltd.

3′ ends of an outer forward block nucleic acid and an outer reverse block nucleic acid were both modified by phosphate groups.

dNTP was purchased from Invitrogen Corp. Bioanalyzer 2100 (manufactured by Agilent Technologies, Inc.) was used for electrophoresis analysis after PCR.

In the following Example 1, Comparative Example 1a, and Comparative Example 1b, the target sequence was a DNA fragment included in the human ABO blood group gene.

### Comparative Example 1a

In the present Comparative Example 1a, the sequence of the outer forward primer was 5′-GCCAGCTCCATGTGGCCGCAC-3′ (SEQ ID NO (sequence identification number): 1; “ABO-OF” hereinafter). The sequence of the outer reverse primer was 5′-CCTGGGTCTCTACCCTCGGC-3′ (SEQ ID NO: 2; “ABO-OR” hereinafter). This primer pair amplifies a 210 bp DNA fragment included in the ABO blood group gene of a human having type AB blood type. This primer pair amplifies a 209 bp DNA fragment included in the ABO blood group gene of a human having type O blood type.

The sequence of the inner forward primer was 5′-TGCAGTAGGAAGGATGTCCTC-3′ (SEQ ID NO: 3; “ABO-IF” hereinafter). The sequence of the inner reverse primer was 5′-TTCTTGATGGCAAACACAGTTAAC-3′ (SEQ ID NO: 4; “ABO-IR” hereinafter). This primer pair of ABO-IF and ABO-IR amplifies a 140 bp DNA fragment (in the case of blood type of AB. In the case of blood type O, a 139 bp DNA fragment) that exists within the 210 bp DNA fragment. By using these primers, a nested PCR was conducted as described in the following.

Described in the following is the composition of the PCR solution for the first stage.

1× TITANIUM Taq DNA polymerase (Produced by Clontech Laboratories, Inc.),

1× TITANIUM Taq PCR Buffer (Produced by Clontech Laboratories, Inc.),

200 μM dNTP,

1 μM ABO-OF,

1 μM ABO-OR, 0.5 ng/μL

5 ng/μl genomic DNA (type AB test subject origin)

Total volume: 10 μL

PCR temperature profiles 1A to 1C are as described in Table 1.

TABLE 1

| | |
|---|---|
| Temperature, Time | Number of Cycles |

Profile 1A

| | | | |
|---|---|---|---|
| Upon Initiation | 95° C., | 1 minute | 1 cycle |
| First Stage | 95° C., | 1 second | 5 cycles |
| | 53.5° C., | 1 second | |
| | 72° C., | 1 second | |
| Second Stage | 95° C., | 1 second | 45 cycles |
| | 53.5° C., | 1 second | |
| | 72° C., | 1 second | |

Profile 1B

| | | | |
|---|---|---|---|
| Upon Initiation | 95° C., | 1 minute | 1 cycle |
| First Stage | 95° C., | 1 second | 10 cycles |
| | 53.5° C., | 1 second | |
| | 72° C., | 1 second | |
| Second Stage | 95° C., | 1 second | 40 cycles |
| | 53.5° C., | 1 second | |
| | 72° C., | 1 second | |

Profile 1C

| | | | |
|---|---|---|---|
| Upon Initiation | 95° C., | 1 minute | 1 cycle |
| First Stage | 95° C., | 1 second | 20 cycles |
| | 53.5° C., | 1 second | |
| | 72° C., | 1 second | |
| Second Stage | 95° C., | 1 second | 30 cycles |
| | 53.5° C., | 1 second | |
| | 72° C., | 1 second | |

The PCR solution of the second step was prepared by adding 0.5 μL of 20 μM ABO-IF and 0.5 μL, of 20 μM ABO-IR to the reaction solution after the first stage of PCR.

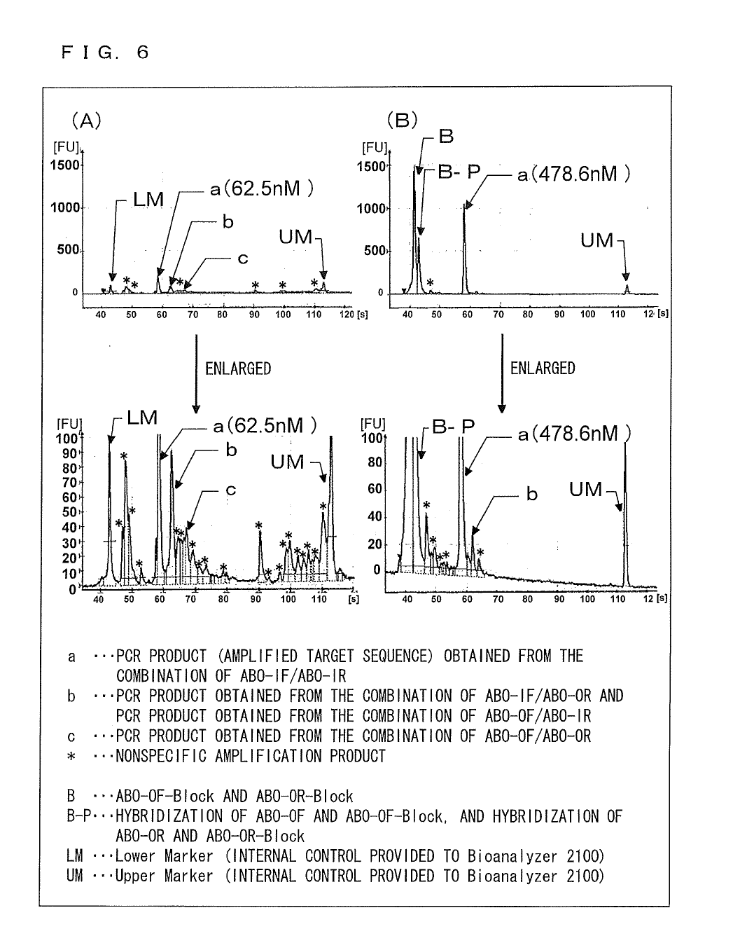

FIG. 6 (A) shows an electrophoresis analysis result from profile 1A. FIG. 6 (A) shows detection of not only a DNA fragment (i.e., target sequence) obtained from the combination of ABO-IF and ABO-IR, but detection of also an undesired DNA fragment obtained from the combination of ABO-OF and ABO-OR. Furthermore, other than peaks of these amplification products, FIG. 6 (A) shows a large number of peaks indicating nonspecific amplification products. The concentration of the DNA fragment obtained from the combination of ABO-IF and ABO-IR was 62.5 nM.

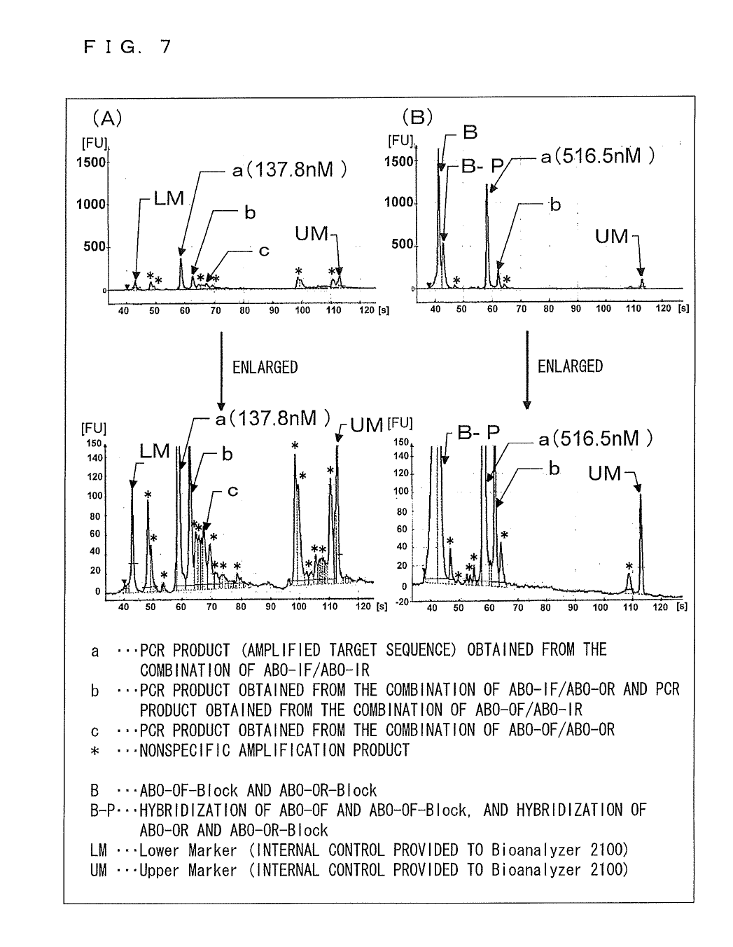

FIG. 7 (A) shows an electrophoresis analysis result from profile 1B. Similar to FIG. 6 (A), FIG. 7 (A) also shows a large number of peaks indicating nonspecific amplification products. The concentration of the DNA fragment obtained from the combination of ABO-IF and ABO-IR was 137.8 nM.

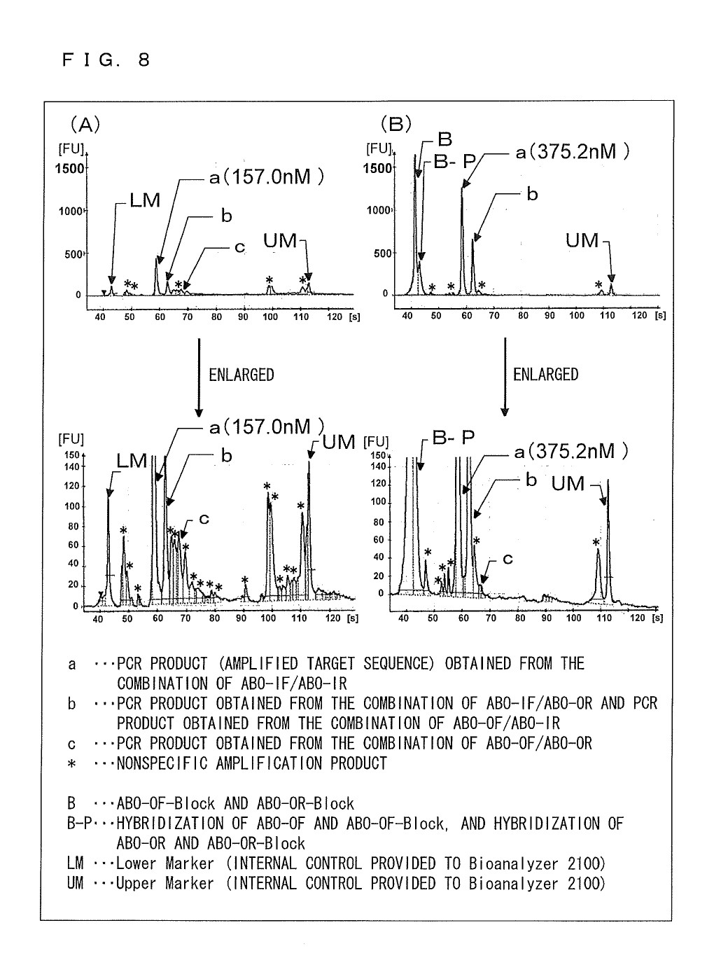

FIG. 8 (A) shows an electrophoresis analysis result from profile 1C. Similar to FIG. 6 (A), FIG. 8 (A) also shows a large number of peaks indicating nonspecific amplification products. The concentration of the DNA fragment obtained from the combination of ABO-IF and ABO-IR was 157.0 nM.

### Example 1

In the present Example 1, the outer forward primer (ABO-OF), the outer reverse primer (ABO-OR), the inner forward primer (ABO-IF), and the inner reverse primer (ABO-IR), which are identical to those in Comparative Example 1a, were used. By using these primers, a nested PCR was conducted as described in the following.

Described in the following is the composition of the PCR solution for the first stage, which is completely identical to that of Comparative Example 1a.

1× TITANIUM Taq DNA polymerase,

1× TITANIUM Taq PCR Buffer

200 μM dNTP

1 μM ABO-OF

1 μM ABO-OR

0.5 ng/μL genomic DNA

Total volume: 10 μL

Temperature profile 1 was similar to that of Comparative Example 1a.

The PCR solution of the second step was prepared by adding

0.5 μL of 20 μM ABO-IF

0.5 μL of 20 μM ABO-IR

1 μL of the outer forward block nucleic acid, and

1 μL of the outer reverse block nucleic acid

to the reaction solution after the first stage of PCR.

The outer forward block nucleic acid was an oligo DNA (hereinafter, “ABO-OF-Block”) which is in a concentration of 100 μM and which consists of a sequence of 5′-GTGCGGCCACATGGAGCTGGC-3′ (SEQ ID NO: 5) and in which the 3′ end thereof was modified via phosphorylation. This sequence was complementary to ABO-OF.

The outer reverse block nucleic acid was a 100 μM oligo DNA (hereinafter, “ABO-OR-Block”) which consists of a sequence of 5′-GCCGAGGGTAGAGACCCAGG-3′ (SEQ ID NO: 6), and in which the 3′ end thereof was modified via phosphorylation. This sequence was complementary to ABO-OR.

FIG. 6 (B) shows an electrophoresis analysis result from profile 1A. FIG. 6 (B) shows detection of the DNA fragment (i.e., target sequence) obtained from the combination of ABO-IF and ABO-IR, but almost no detection of the DNA fragment obtained from the combination of ABO-OF and ABO-OR. Furthermore, FIG. 6 (B) shows almost no peaks indicating nonspecific amplification products. The concentration of the DNA fragment obtained from the combination of ABO-IF and ABO-IR was 478.6 nM. As can be understood from FIG. 6 (B), since the PCR solution of the second step contained ABO-OF-Block and ABO-OR-Block, the PCR with the combination of ABO-IF and ABO-IR was conducted extremely efficiently, and nonspecific amplifications were significantly inhibited.

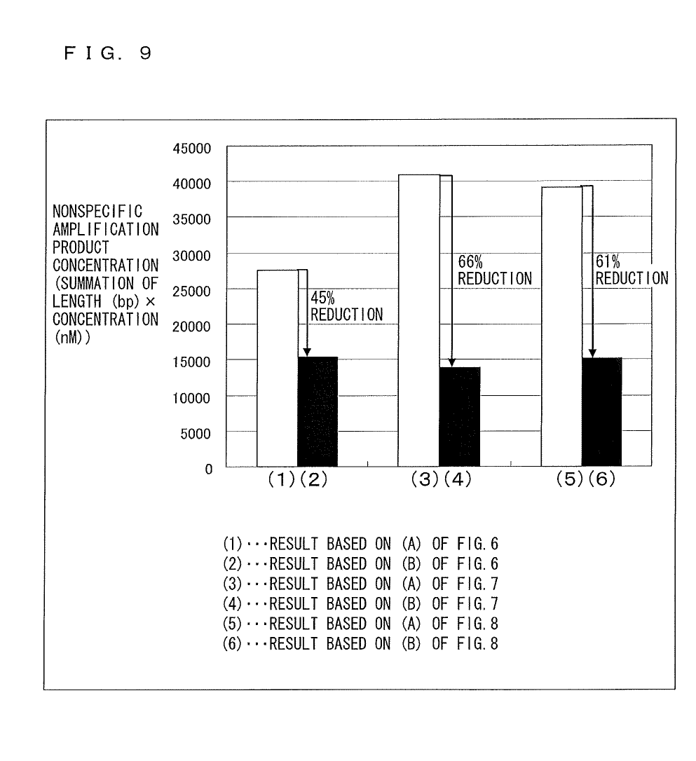

FIG. 9 quantitatively shows the inhibition of nonspecific amplifications. These are bar graphs obtained by calculating concentrations of pyrophosphoric acid produced upon amplification of all the nonspecific amplification products detected by electrophoresis analysis.

In a DNA extension reaction, it is well known that one molecule of pyrophosphoric acid is produced every time the extension reaction proceeds for a single base. A concentration of the pyrophosphoric-acid that is produced when all the nonspecific amplification products are amplified can be obtained by, calculating length (bp)×concentration (nM) for each DNA fragment that is amplified nonspecifically, and adding up all of those. The length and concentration of each of the DNA fragments can be obtained by Bioanalyzer 2100.

(1) and (2) of FIG. 9 show that the nested PCR shown in FIG. 6 (B) can inhibit about 45% of the nonspecific amplifications when compared to the nested PCR result shown in FIG. 6 (A). The concentration of DNA amplified can be measured by measuring the concentration of pyrophosphoric acid contained in the solution after the reaction. The pyrophosphoric acid produced from nonspecific amplifications can be a large noise. However, an addition of a block nucleic acid can reduce the noise originating from the pyrophosphoric acid produced from nonspecific amplifications.

FIG. 7 (B) shows an electrophoresis analysis result from profile 1B. The target sequence was detected similarly as in FIG. 6 (B), however, FIG. 7 (B) shows almost no peaks indicating nonspecific amplification products, including the DNA fragment obtained from the combination of ABO-OF and ABO-OR. The concentration of the DNA fragment obtained from the combination of ABO-IF and ABO-IR was 516.5 nM.

(3) and (4) of FIG. 9 show that the nested PCR shown in FIG. 7 (B) can inhibit about 66% of the nonspecific amplifications when compared to the nested PCR result shown in FIG. 7 (A).

FIG. 8 (B) shows an electrophoresis analysis result from profile 1C. The target sequence was detected similarly as in FIG. 6 (B), however, FIG. 8 (B) shows almost no peaks indicating nonspecific amplification products, including the DNA fragment obtained from the combination of ABO-OF and ABO-OR. The concentration of the DNA fragment obtained from the combination of ABO-IF and ABO-IR was 375.2 nM.

(5) and (6) of FIG. 9 show that the nested PCR shown in FIG. 8(B) can inhibit about 61% of the nonspecific amplifications when compared to the nested PCR result shown in FIG. 8(A).

Example 1 and Comparative Example 1a show that the addition of the block nucleic acid can significantly increase efficiency of amplification of the single stranded target sequence, and greatly inhibit nonspecific amplifications.

For confirmation, the inventors of the present invention conducted an experiment by adding only ABO-OF-Block after the first stage of PCR. As a result, even in this case, it was confirmed that efficiency of amplification of the target sequence increases and nonspecific amplifications are inhibited when compared to the case where neither of ABO-OF-Block and ABO-OR-Block were added, although not to a degree of the case where both ABO-OF-Block and ABO-OR-Block were added.

### Comparative Example 1b

As in the case of FIG. 4, used in Comparative Example 1b were ABO-OF-Block (“outer forward primer” in FIG. 4) in which the 3′ end thereof have not been modified via phosphorylation, and ABO-OR-Block (“outer reverse primer” of FIG. 4) in which the 3′ end thereof have not been modified via phosphorylation. Profile 1A was used.

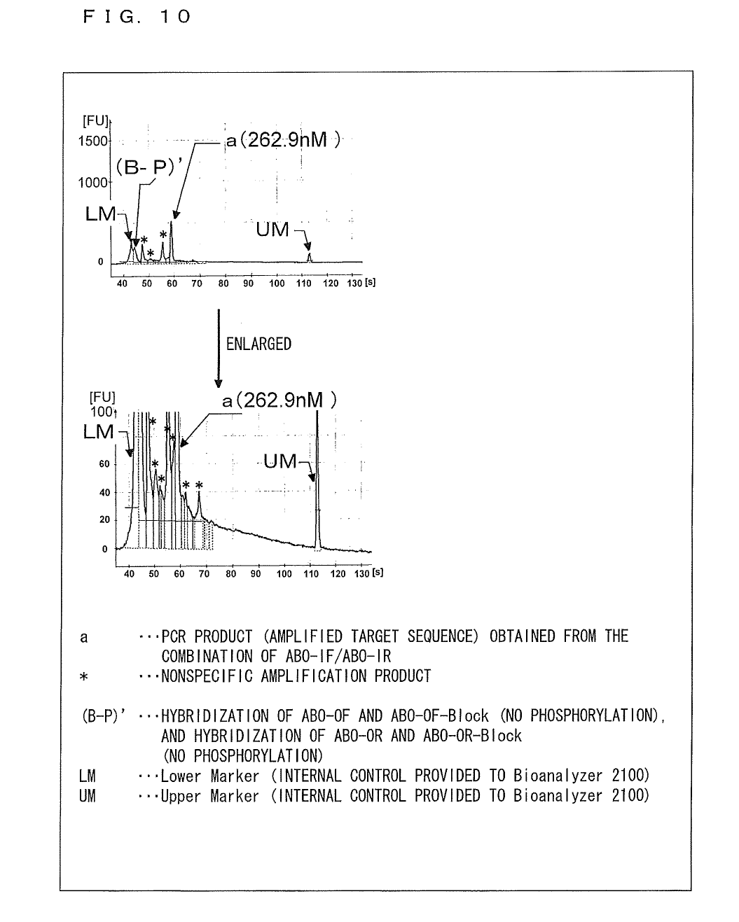

FIG. 10 shows a result of the electrophoresis. The concentration of the DNA fragment obtained from the combination of ABO-IF and ABO-IR in FIG. 10 is obviously smaller than that in FIG. 6 (B). Furthermore, the effect on inhibiting nonspecific amplifications in FIG. 10 is obviously smaller than that in FIG. 6 (B).

Therefore, the block primer in which the 3′ end thereof have not been modified via phosphorylation could not obtain a sufficient effect on inhibiting nonspecific amplifications.

The following Example 2 and Comparative Example 2 correspond to FIG. 5. In the following Example 2 and Comparative Example 2, the target sequence was a DNA fragment included in the human ALDH2 gene.

### Comparative Example 2

In the present Comparative Example 2, the sequence of the outer forward primer was 5′-CAAATTACAGGGTCAACTGCT-3′ (SEQ ID NO: 7; “ALDH2-OF” hereinafter). The sequence of the outer reverse primer was 5′-GGCAGGTCCTGAACCTC-3′ (SEQ ID NO: 8; “ALDH2-OR” hereinafter). This primer pair amplifies a 251 bp DNA fragment included in the human ALDH2 gene.

The sequence of the inner forward primer was 5′-GTACGGGCTGCAGGCATACAC-3′ (SEQ ID NO: 9; “ALDH2-IF” hereinafter). The sequence of the inner reverse primer is identical to that of ALDH2-OR. The primer pair of ALDH2-IF and ALDH2-OR can amplify a 160 bp DNA fragment that exists within the 251 bp DNA fragment. By using these primers, a nested PCR was conducted as described in the following.

Described in the following is the composition of the PCR solution for the first stage.

0.05 U/μL TaKaRa LA Taq HS (Produced by Takara Bio Inc.),

1× LA PCR Buffer II (Mg²⁺ plus) (Produced by Takara Bio Inc.),

200 μM dNTP,

1 μM ALDH2-OF,

1 μM ALDH2-OR,

0.83 ng/μL genomic DNA

Total volume: 10 μL

PCR temperature profiles 2A to 2C are described in Table 2.

TABLE 2

| | |
|---|---|
| Temperature, Time | Number of Cycles |

Profile 2A

| | | | |
|---|---|---|---|
| Upon Initiation | 95° C., | 1 minute | 1 cycle |
| First Stage | 95° C., | 10 seconds | 5 cycles |
| | 59.8° C., | 10 seconds | |
| | 72° C., | 30 seconds | |
| Second Stage | 95° C., | 10 seconds | 25 cycles |
| | 59.8° C., | 10 seconds | |
| | 72° C., | 30 seconds | |

Profile 2B

| | | | |
|---|---|---|---|
| Upon Initiation | 95° C., | 1 minute | 1 cycle |
| First Stage | 95° C., | 10 seconds | 10 cycles |
| | 59.8° C., | 10 seconds | |
| | 72° C., | 30 seconds | |
| Second Stage | 95° C., | 10 seconds | 20 cycles |
| | 59.8° C., | 10 seconds | |
| | 72° C., | 30 seconds | |

Profile 2C

| | | | |
|---|---|---|---|
| Upon Initiation | 95° C., | 1 minute | 1 cycle |
| First Stage | 95° C., | 10 seconds | 20 cycles |
| | 59.8° C., | 10 seconds | |
| | 72° C., | 30 seconds | |
| Second Stage | 95° C., | 10 seconds | 30 cycles |
| | 59.8° C., | 10 seconds | |
| | 72° C., | 30 seconds | |

The PCR solution of the second step was prepared by adding 1 μL of 10 μM ALDH2-IF to the reaction solution after the first stage of PCR.

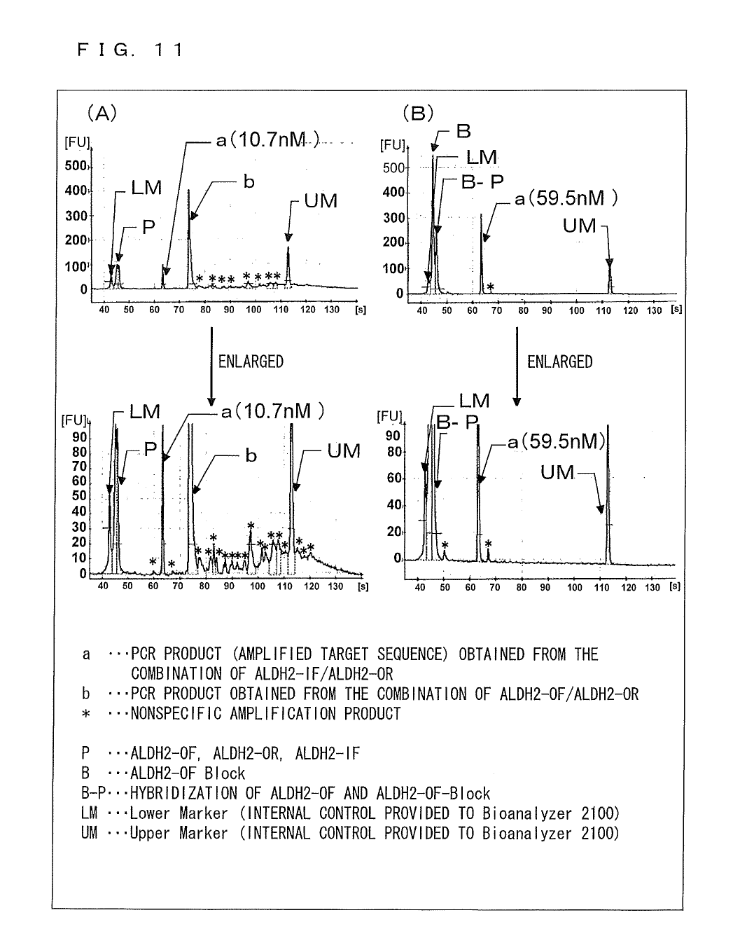

FIG. 11 (A) shows an electrophoresis analysis result from profile 2A. FIG. 11 (A) shows detection of not only a DNA fragment (i.e., target sequence) obtained from the combination of ALDH2-IF and ALDH2-IR, but detection of also a DNA fragment obtained from the combination of ALDH2-OF and ALDH2-OR. Furthermore, FIG. 11 (A) shows a large number of peaks indicating nonspecific amplification products. The concentration of the DNA fragment obtained from the combination of ALDH2-IF and ALDH2-IR was 10.7 nM.

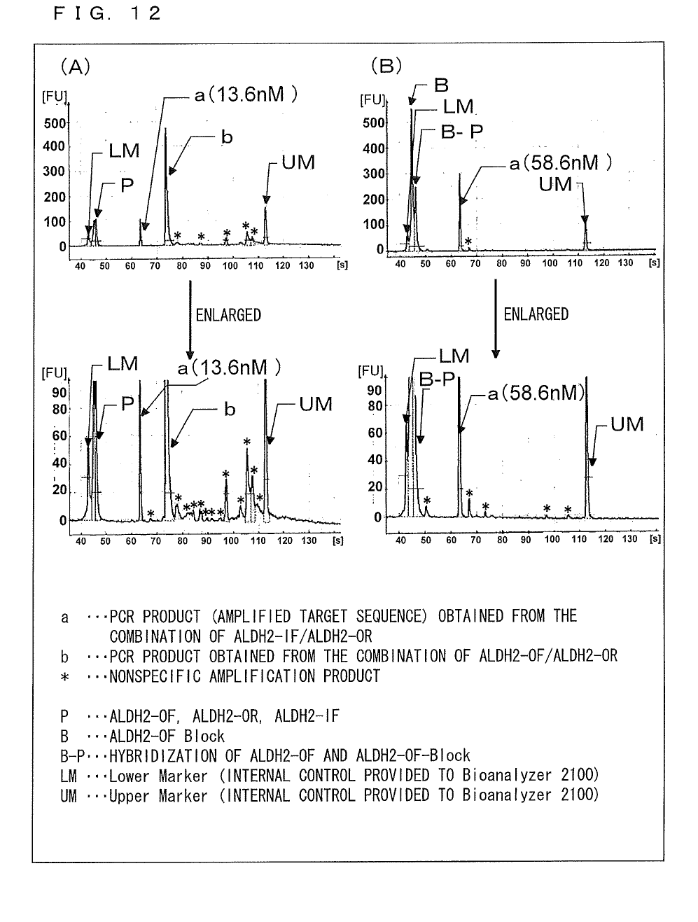

FIG. 12 (A) shows an electrophoresis analysis result from profile 2B. Similar to FIG. 11 (A), FIG. 12 (A) also shows a large number of peaks indicating nonspecific amplification products. The concentration of the DNA fragment obtained from the combination of ALDH2-IF and ALDH2-IR was 13.6 nM.

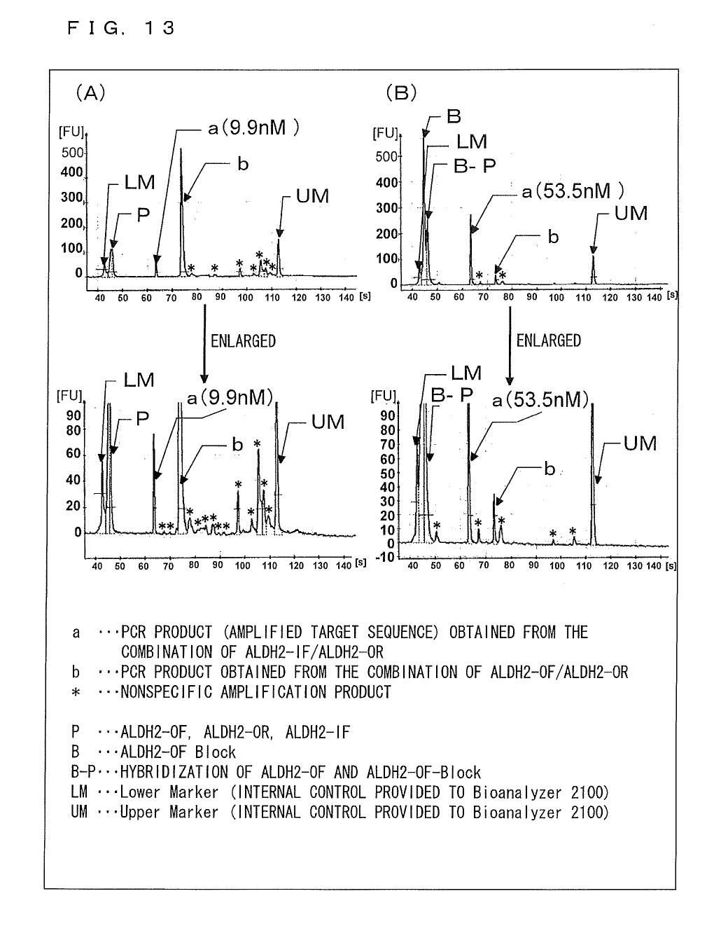

FIG. 13 (A) shows an electrophoresis analysis result from profile 2C. Similar to FIG. 11 (A), FIG. 13 (A) also shows a large number of peaks indicating nonspecific amplification products. The concentration of the DNA fragment obtained from the combination of ALDH2-IF and ALDH2-IR was 9.9 nM.

### Example 2

Used in Experimental Example 2 was a PCR solution obtained by adding

1 μL of 10 μM ALDH2-IF, and

1 μL of the outer forward block nucleic acid

to the reaction solution of the first stage of PCR.

The outer forward block nucleic acid was an oligo DNA (hereinafter, “ALDH2-OF-Block”) which is in a concentration of 100 μM and which consists of a sequence of 5′-AGCAGTTGACCCTGTAATTTG-3′ (SEQ ID NO: 10) and in which the 3′ end thereof was modified via phosphorylation. This sequence was complementary to ALDH2-OF.

FIG. 11 (B) shows an electrophoresis analysis result from profile 2A. FIG. 11 (B) shows detection of the DNA fragment (i.e., target sequence) obtained from the combination of ALDH2-IF and ALDH2-IR, but almost no detection of the DNA fragment obtained from the combination of ALDH2-OF and ALDH2-OR. Furthermore, FIG. 11 (B) shows almost no peaks indicating nonspecific amplification products. The concentration of the DNA fragment obtained from the combination of ALDH2-IF and ALDH2-IR was 58.6 nM. As can be understood from FIG. 11 (B), since the PCR solution of the second step contained ALDH2-OF-Block and ALDH2-OR-Block, the PCR with the combination of ALDH2-IF and ALDH2-IR was conducted extremely efficiently, and nonspecific amplifications were significantly inhibited.

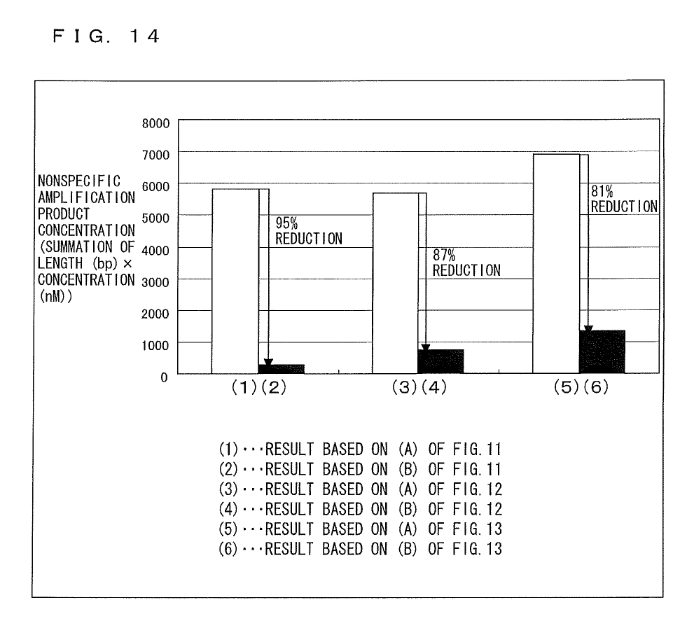

Similar to FIG. 9, FIG. 14 quantitatively shows the inhibition of nonspecific amplifications. (1) and (2) of FIG. 14 show that the nested PCR shown in FIG. 11 (B) can inhibit about 95% of the nonspecific amplifications when compared to the nested PCR result shown in FIG. 11 (A).

FIG. 12 (B) shows an electrophoresis analysis result from profile 2B. The target sequence was detected similarly as in FIG. 11 (B), however, FIG. 12 (B) shows almost no peaks indicating nonspecific amplification products, including the DNA fragment obtained from the combination of ALDH2-OF and ALDH2-OR. The concentration of the DNA fragment obtained from the combination of ALDH2-IF and ALDH2-IR was 58.6 nM.

(3) and (4) of FIG. 14 show that the nested PCR shown in FIG. 12 (B) can inhibit about 87% of the nonspecific amplifications when compared to the nested PCR result shown in FIG. 12 (A).

FIG. 13 (B) shows an electrophoresis analysis result from profile 2C. The target sequence was detected similarly as in FIG. 11 (B), however, FIG. 13 (B) shows almost no peaks indicating nonspecific amplification products, including the DNA fragment obtained from the combination of ALDH2-OF and ALDH2-OR. The concentration of the DNA fragment obtained from the combination of ALDH2-IF and ALDH2-IR was 53.5 nM.

(5) and (6) of FIG. 14 show that the nested PCR shown in FIG. 13 (B) can inhibit about 81% of the nonspecific amplifications when compared to the nested PCR result shown in FIG. 13 (A).

Example 2 and Comparative Example 2 show that the addition of the block nucleic acid can increase efficiency of amplification of the target sequence, and inhibit nonspecific amplifications.

The following Example 3, Comparative Example 3a, and Comparative Example 3b correspond to FIG. 3. In the following Example 3, Comparative Example 3a, and Comparative Example 3b, the target sequence was a DNA fragment included in the human dystrophin gene.

### Comparative Example 3a

In the present Comparative Example 3a, the sequence of the outer forward primer was 5′-GATGGCAAAAGTGTTGAGAAAAAGTC-3′ (SEQ ID NO: 11; “DYSTRO-OF” hereinafter). The sequence of the outer reverse primer was 5′-TTCTACCACATCCCATTTTCTTCCA-3′ (SEQ ID NO: 12; “DYSTRO-OR” hereinafter). This primer pair can amplify a 459 bp DNA fragment included in the human dystrophin gene.

The sequence of the inner forward primer was 5′-AGGCTTGAAAGGGCAAGTAGAAGT-3′ (SEQ ID NO: 13; “DYSTRO-IF” hereinafter). The sequence of the inner reverse primer was 5′-GCTGATCTGCTGGCATCTTGC-3′ (SEQ ID NO: 14; “DYSTRO-IR” hereinafter). This primer pair of DYSTRO-IF and DYSTRO-IR can amplify a 147 bp DNA fragment that exists within the 459 bp DNA fragment. By using these primers, a nested PCR was conducted as described in the following.

Described in the following is the composition of the PCR solution for the first stage.

1× TITANIUM Taq DNA polymerase (Produced by Clontech Laboratories, Inc.),

1× TITANIUM Taq PCR Buffer (Produced by Clontech Laboratories, Inc.),

200 μM dNTP,

1 μM DYSTRO-OF,

1 μM DYSTRO-OR,

0.5 ng/μL, genomic DNA

Total volume: 10 μL

PCR temperature profiles 3A to 3B are described in Table 3.

TABLE 3

| | |
|---|---|
| Temperature, Time | Number of Cycles |

Profile 3A

| | | | |
|---|---|---|---|
| Upon Initiation | 95° C., | 1 minute | 1 cycle |
| First Stage | 95° C., | 1 second | 5 cycles |
| | 56.8° C., | 1 second | |
| | 72° C., | 1 second | |
| Second Stage | 95° C., | 1 second | 45 cycles |
| | 56.8° C., | 1 second | |
| | 72° C., | 1 second | |

Profile 3B

| | | | |
|---|---|---|---|
| Upon Initiation | 95° C., | 1 minute | 1 cycle |
| First Stage | 95° C., | 1 second | 15 cycles |
| | 56.8° C., | 1 second | |
| | 72° C., | 1 second | |
| Second Stage | 95° C., | 1 second | 35 cycles |
| | 56.8° C., | 1 second | |
| | 72° C., | 1 second | |

The PCR solution of the second step was prepared by adding 0.5 μL of 20 μM DYSTRO-IF and 0.5 μL of 20 μM DYSTRO-IR to the reaction solution after the first stage of PCR.

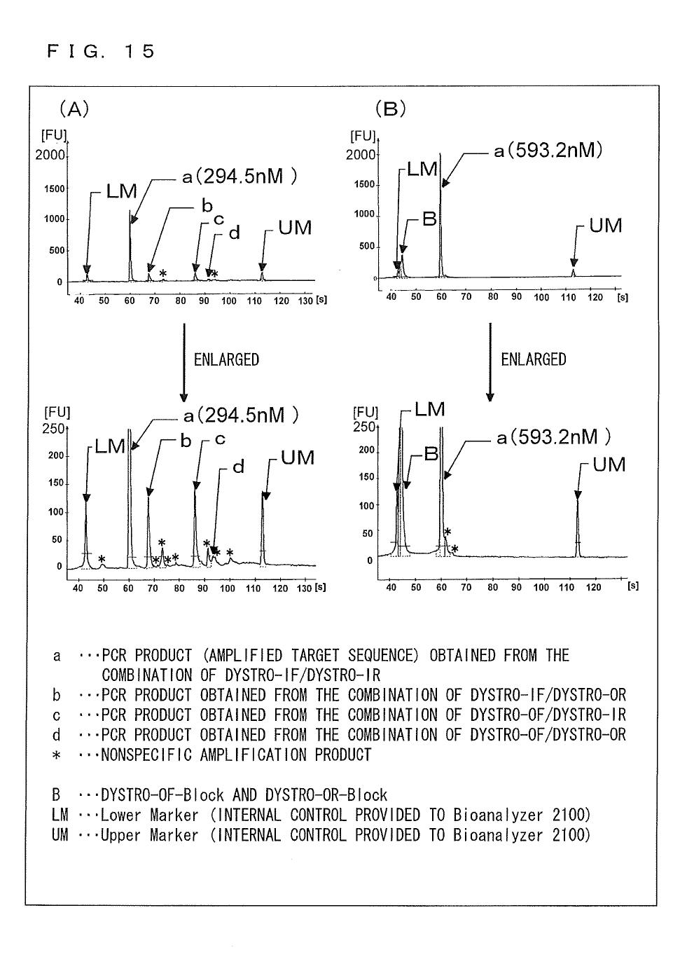

FIG. 15 (A) shows an electrophoresis analysis result from profile 3A. FIG. 15 (A) shows detection of not only a DNA fragment (i.e., target sequence) obtained from the combination of DYSTRO-IF and DYSTRO-IR, but detection of also a DNA fragment obtained from the combination of DYSTRO-OF and DYSTRO-OR. Furthermore, other than peaks of these amplification products, FIG. 15 (A) shows a large number of peaks indicating nonspecific amplification products. The concentration of the DNA fragment obtained from the combination of DYSTRO-IF and DYSTRO-IR was 294.5 nM.

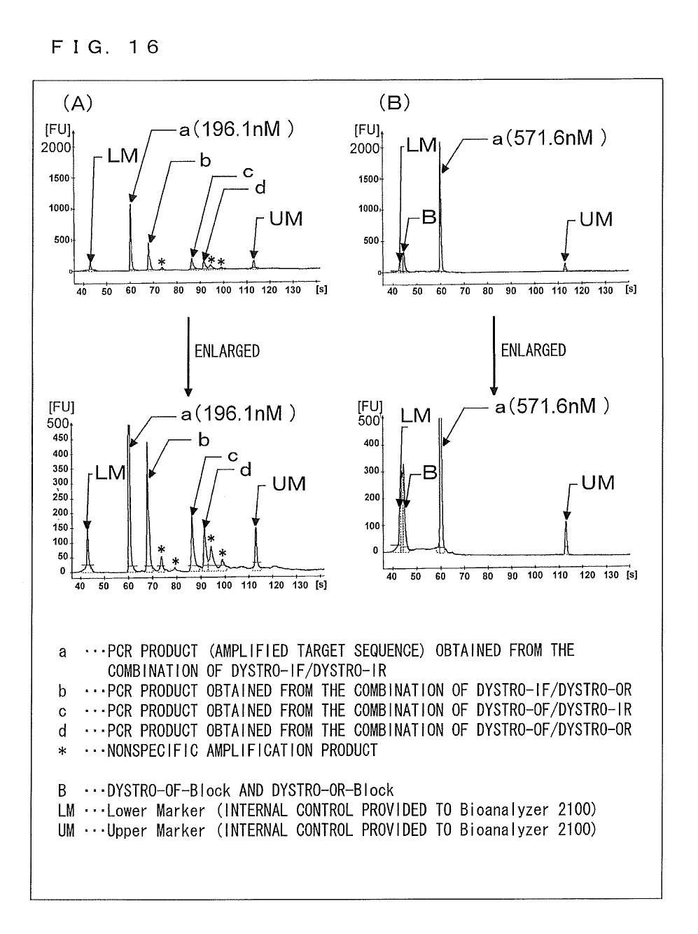

FIG. 16 (A) shows an electrophoresis analysis result from profile 3B. Similar to FIG. 15 (A), FIG. 16 (A) also shows a large number of peaks indicating nonspecific amplification products. The concentration of the DNA fragment obtained from the combination of DYSTRO-IF and DYSTRO-IR was 196.1 nM.

### Example 3

In the present Example 3, the outer forward primer (DYSTRO-OF), the outer reverse primer (DYSTRO-OR), the inner forward primer (DYSTRO-IF), and the inner reverse primer (DYSTRO-IR), which are identical to those in Comparative Example 3a, were used. By using these primers, a nested PCR was conducted as described in the following.

The composition of the PCR solution and temperature profiles 3A•3B for the first stage were completely identical to those of Comparative Example 3a.

The PCR solution of the second step was prepared by adding

0.5 μL of 20 μM DYSTRO-IF

0.5 μL of 20 μM DYSTRO-IR

1 μL of the outer forward block nucleic acid, and

1 μL of the outer reverse block nucleic acid

to the reaction solution after the first stage of PCR.

The outer forward block nucleic acid was a 100 μM oligo DNA (hereinafter, “DYSTRO-OF-Block”) which consists of a sequence of 5′-GACTTTTTCTCAACACTYFTGCCATC-3′ (SEQ ID NO: 15) and in which the 3′ end thereof was modified via phosphorylation. This sequence was complementary to DYSTRO-OF.

The outer reverse block nucleic acid was a 100 μM oligo DNA (hereinafter, “DYSTRO-OR-Block”) which consists of a sequence of 5′-TGGAAGAAAATGGGATGTGGTAGAA-3′ (SEQ ID NO: 16) and in which the 3′ end thereof was modified via phosphorylation. This sequence was complementary to DYSTRO-OR.

FIG. 15 (B) shows an electrophoresis analysis result from profile 3A. As can be understood from FIG. 15 (B), the concentration of the amplification product from the combination DYSTRO-OF and DYSTRO-OR, the concentration of the amplification product from the combination of DYSTRO-OF and DYSTRO-IR, and the concentration of the amplification product from the combination of DYSTRO-IF and DYSTRO-OR were all below detection limit.

The concentration of the DNA fragment obtained from the combination of DYSTRO-IF and DYSTRO-IR was 593.2 nM. As can be understood from FIG. 15 (B), since the PCR solution of the second step contained DYSTRO-OF-Block and DYSTRO-OR-Block, the PCR with the combination of DYSTRO-IF and DYSTRO-IR was conducted extremely efficiently, and nonspecific amplifications were significantly inhibited.

FIG. 16 (B) shows an electrophoresis analysis result from profile 3B. As can be understood from FIG. 16 (B), the concentration of the amplification product from the combination of DYSTRO-OF and DYSTRO-OR, the concentration of the amplification product from the combination of DYSTRO-OF and DYSTRO-IR, and the concentration of the amplification product from the of the combination of DYSTRO-IF and DYSTRO-OR were all below detection limits.

The concentration of the DNA fragment obtained from the combination of DYSTRO-IF and DYSTRO-IR was 571.6 nM. As can be understood from FIG. 16 (B), since the PCR solution of the second step contained DYSTRO-OF-Block and DYSTRO-OR-Block, the PCR with the combination of DYSTRO-IF and DYSTRO-IR was conducted extremely efficiently, and nonspecific amplifications were significantly inhibited.

### Comparative Example 3b

As in the case of FIG. 4, used in Comparative Example 3b were DYSTRO-OF-Block (“outer forward primer” of FIG. 4) in which the 3′ end thereof was not modified via phosphorylation, and DYSTRO-OR-Block (“outer reverse primer” in FIG. 4) in which the 3′ end thereof was not modified via phosphorylation. Profile 3A was used.

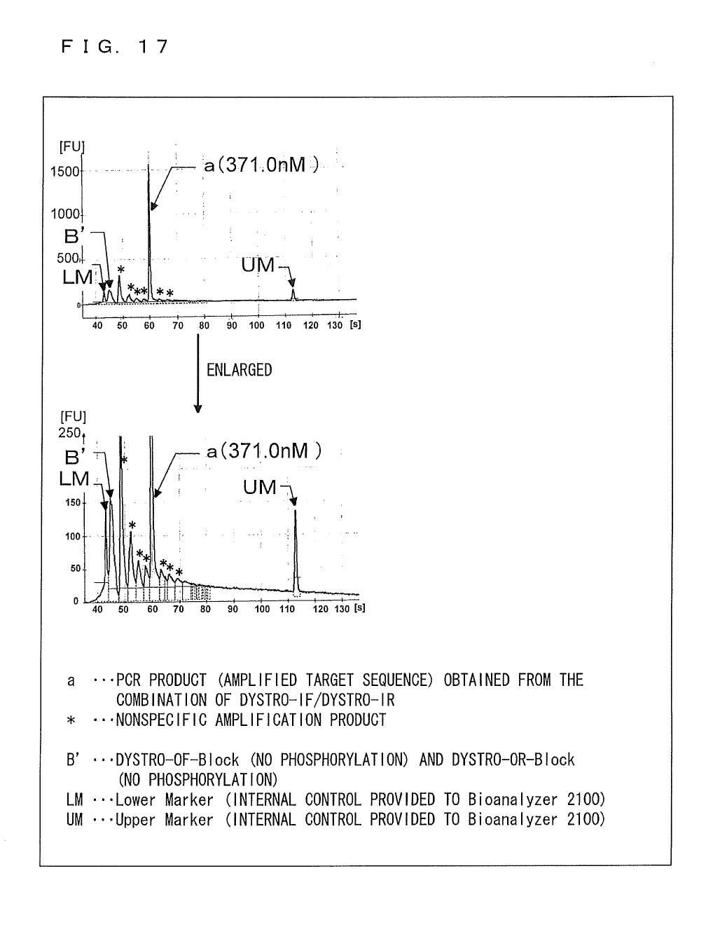

FIG. 17 shows a result of the electrophoresis. The concentration of the DNA fragment obtained from the combination of DYSTRO-IF and DYSTRO-IR in FIG. 17 is obviously smaller than those in FIG. 15 (B) and FIG. 16 (B). Furthermore, the effect on inhibiting nonspecific amplifications in FIG. 17 is obviously smaller than those of FIG. 15 (B) and FIG. 16 (B).

Therefore, the block primer in which the 3′ end thereof was not modified via phosphorylation could not obtain a sufficient effect on inhibiting nonspecific amplifications.

### INDUSTRIAL APPLICABILITY

The present invention provides a method for amplifying a target sequence, and the method for amplifying the target sequence demonstrates high efficiency of amplification of the target sequence and a significant effect on inhibiting nonspecific amplifications.

### DESCRIPTION OF THE REFERENCE CHARACTERS

1 target sequence
1a single stranded target sequence
1b complementary single stranded target sequence
4of outer forward primer
4ofb outer forward block nucleic acid
4if inner forward primer
5or outer reverse primer
5ir inner reverse primer
5orb outer reverse block nucleic acid
6 first single stranded DNA
6a first unamplified sequence
6b second unamplified sequence
6c third unamplified sequence
6d fourth unamplified sequence
6m intermediate single stranded target sequence
6s sequence portion identical or similar to a sequence complementary to outer reverse block primer
7 second single stranded DNA
7a fifth unamplified sequence
7b sixth unamplified sequence
7c seventh unamplified sequence
7d eighth unamplified sequence
7m complementary single stranded intermediate target sequence
7s sequence portion identical or similar to a sequence complementary to outer forward block primer

### SEQUENCE LISTING FREE TEXT

SEQ ID NO: 1, outer forward primer for ABO blood group gene;

SEQ ID NO: 2, outer reverse primer for ABO blood group gene;

SEQ ID NO: 3, inner forward primer for ABO blood group gene;

SEQ ID NO: 4, inner reverse primer for ABO blood group gene;

SEQ ID NO: 5, block nucleic acid (DNA) of outer forward primer for ABO blood group gene;

SEQ ID NO: 6, block nucleic acid (DNA) of outer reverse primer for ABO blood group gene;

SEQ ID NO: 7, outer forward primer for ALDH2 gene;

SEQ ID NO: 8, outer reverse primer or inner reverse primer for ALDH2 gene;

SEQ ID NO: 9, inner forward primer for ALDH2 gene;

SEQ ID NO: 10, block nucleic acid (DNA) of outer forward primer for ALDH2 gene;

SEQ ID NO: 11, outer forward primer for dystrophin gene;

SEQ ID NO: 12, outer reverse primer for dystrophin gene;

SEQ ID NO: 13, inner forward primer for dystrophin gene;

SEQ ID NO: 14, inner reverse primer for dystrophin gene;

SEQ ID NO: 15, block nucleic acid (DNA) of outer forward primer for dystrophin gene; and

SEQ ID NO: 16, block nucleic acid (DNA) of outer reverse primer for dystrophin gene.

What is claimed is:

1. A method for amplifying a double stranded target sequence (1) in a double stranded DNA consisting of a first single stranded DNA (6) and a second single stranded DNA (7), wherein
the double stranded target sequence (1) consists of a single stranded target sequence (1a) and a complementary single stranded target sequence (1b),
the first single stranded DNA (6) consists of a 3′ end—a first unamplified sequence (6a)—a second unamplified sequence (6b)—the single stranded target sequence (1a)—a third unamplified sequence (6c)—a fourth unamplified sequence (6d)—a 5′ end,
the second single stranded DNA (7) consists of a 5′ end—a fifth unamplified sequence (7a)—a sixth unamplified sequence (7b)—the complementary single stranded target sequence (1b)—a seventh unamplified sequence (7c)—an eighth unamplified sequence (7d)—a 3′ end, and
the complementary single stranded target sequence (1b), the fifth unamplified sequence (7a), the sixth unamplified sequence (7b), the seventh unamplified sequence (7c), and the eighth unamplified sequence (7d) are complementary to the single stranded target sequence (1a), the first unamplified sequence (6a), the second unamplified sequence (6b), the third unamplified sequence (6c), and the fourth unamplified sequence (6d), respectively, and
the method comprising the following step (A) and step (B):
the step (A) of mixing DNA polymerase, deoxynucleoside triphosphate, the double stranded DNA (6•7), an outer forward primer (4of), and an outer reverse primer (5or) to amplify an intermediate double stranded DNA by utilizing a polymerase chain reaction, wherein
the intermediate double stranded DNA consists of an intermediate target sequence and a complementary intermediate target sequence,
the intermediate target sequence consists of a 3′ end—the second unamplified sequence (6b)—the single stranded target sequence (1a)—the third unamplified sequence (6c)—a 5′ end,
the complementary intermediate target sequence consists of a 5′ end—the sixth unamplified sequence (7b)—the complementary single stranded target sequence (1b)—the seventh unamplified sequence (7c)—a 3′ end,
the outer forward primer (4of) is complementary to a 3′ end sequence portion included in the second unamplified sequence (6b), and
the outer reverse primer (5or) is complementary to a 3′ end sequence portion included in the seventh unamplified sequence; and
the step (B) of mixing DNA polymerase, deoxynucleoside triphosphate, the intermediate double stranded DNA, an inner forward primer (4if), an inner reverse primer (5ir), and an outer forward block nucleic acid (4ofb) to amplify specifically the target sequence (1) by utilizing a polymerase chain reaction, wherein
the inner forward primer (4if) is complementary to a 3′ end sequence portion included in the single stranded target sequence (1a),
the inner reverse primer (5ir) is complementary to a 3′ end sequence portion included in the complementary single stranded target sequence (1b), and
the outer forward block nucleic acid (4ofb) is complementary to the outer forward primer (4of) and unable to be an origin of a DNA extension reaction by the DNA polymerase.

2. The method according to claim 1, wherein
an outer reverse block nucleic acid (5orb) is additionally mixed at the step (B), and
the outer reverse block nucleic acid (5orb) is complementary to the outer reverse primer (5or) and is unable to be an origin of a DNA extension reaction by the DNA polymerase.

3. The method according to claim 1, wherein the outer forward block nucleic acid (4ofb) consists of a DNA in which an OH group at carbon number 3 of a sugar included in a nucleotide located at a 3′ end is substituted or modified by a hydrogen, phosphate group, amino group, biotin group, thiol group, or a derivative thereof.

4. The method according to claim 1, wherein the outer forward block nucleic acid (4ofb) consists of a Locked Nucleic Acid in which an OH group at carbon number 3 of a sugar included in a nucleotide located at a 3′ end is substituted or modified by a hydrogen, phosphate group, amino group, biotin group, thiol group, or a derivative thereof.

5. The method according to claim 1, wherein the outer forward block nucleic acid (4ofb) consists of a Peptide Nucleic Acid.

6. The method according to claim 2, wherein the outer reverse block nucleic acid (5orb) consists of a DNA in which an OH group at carbon number 3 of a sugar included in a nucleotide located at a 3′ end is substituted or modified by a hydrogen, phosphate group, amino group, biotin group, thiol group, or a derivative thereof.

7. The method according to claim 2, wherein the outer reverse block nucleic acid (5orb) consists of a Locked Nucleic Acid in which an OH group at carbon number 3 of a sugar included in a nucleotide located at a 3′ end is substituted or modified by a hydrogen, phosphate group, amino group, biotin group, thiol group, or a derivative thereof.

8. The method according to claim 2, wherein the outer reverse block nucleic acid (5orb) consists of a Peptide Nucleic Acid.

9. A method for amplifying a double stranded target sequence (1) in a double stranded DNA consisting of a first single stranded DNA (6) and a second single stranded DNA (7), wherein
the double stranded target sequence (1) consists of a single stranded target sequence (1a) and a complementary single stranded target sequence (1b),
the first single stranded DNA (6) consists of a 3′ end—a first unamplified sequence (6a)—a second unamplified sequence (6b)—the single stranded target sequence (1a)—a third unamplified sequence (6c)—a fourth unamplified sequence (6d)—a 5′ end,
the second single stranded DNA (7) consists of a 5′ end—a fifth unamplified sequence (7a)—a sixth unamplified sequence (7b)—the complementary single stranded target sequence (1b)—a seventh unamplified sequence (7c)—an eighth unamplified sequence (7d)—a 3′ end, and
the complementary single stranded target sequence (1b), the fifth unamplified sequence (7a), the sixth unamplified sequence (7b), the seventh unamplified sequence (7c), and the eighth unamplified sequence (7d) are complementary to the single stranded target sequence (1a), the first unamplified sequence (6a), the second unamplified sequence (6b), the third unamplified sequence (6c), and the fourth unamplified sequence (6d), respectively, and
the method comprising the following step (A) and step (B):
the step (A) of mixing DNA polymerase, deoxynucleoside triphosphate, the double stranded DNA (6•7), an outer forward primer (4of), and an inner reverse primer (5ir) to amplify an intermediate double stranded DNA by utilizing a polymerase chain reaction, wherein
the intermediate double stranded DNA consists of an intermediate target sequence and a complementary intermediate target sequence, the intermediate target sequence consists of a 3′ end—the second unamplified sequence (6b)—the single stranded target sequence (1a)—a 5′ end,
the complementary intermediate target sequence consists of a 5′ end—the sixth unamplified sequence (7b)—the complementary single stranded target sequence (1b)—a 3′ end,
the outer forward primer (4of) is complementary to a 3′ end sequence portion included in the second unamplified sequence (6b), and
the inner reverse primer (5ir) is complementary to a 3′ end sequence portion included in the complementary single stranded target sequence (1b); and
the step (B) of mixing DNA polymerase, deoxynucleoside triphosphate, the intermediate double stranded DNA, an inner forward primer (4if), and an outer forward block nucleic acid (4ofb) to amplify specifically the target sequence (1) by utilizing a polymerase chain reaction, wherein
the inner forward primer (4if) is complementary to a 3′ end sequence portion included in the single stranded target sequence (1a),
the outer forward block nucleic acid (4ofb) is complementary to the outer forward primer (4of) and unable to be an origin of a DNA extension reaction by the DNA polymerase.

10. The method according to claim 9, wherein the outer forward block nucleic acid (4ofb) consists of a DNA in which an OH group at carbon number 3 of a sugar included in a nucleotide located at a 3′ end is substituted or modified by a hydrogen, phosphate group, amino group, biotin group, thiol group, or a derivative thereof.

11. The method according to claim 9, wherein the outer forward block nucleic acid (4ofb) consists of a Locked Nucleic Acid in which an OH group at carbon number 3 of a sugar included in a nucleotide located at a 3′ end is substituted or modified by a hydrogen, phosphate group, amino group, biotin group, thiol group, or a derivative thereof.

12. The method according to claim 9, wherein the outer forward block nucleic acid (4ofb) consists of a Peptide Nucleic Acid.


SEQUENCE LISTING (16 sequences)

SEQ ID NO: 1 - DNA, 21 bp, Artificial
  Outer Forward primer for ABO gene
gccagctccatgtggccgcac

SEQ ID NO: 2 - DNA, 20 bp, Artificial
  Outer Reverse primer for ABO gene
cctgggtctctaccctcggc

SEQ ID NO: 3 - DNA, 21 bp, Artificial
  Inner Forward Primer for ABO gene
tgcagtaggaaggatgtcctc

SEQ ID NO: 4 - DNA, 24 bp, Artificial
  Inner Reverse Primer for ABO gene
ttcttgatggcaaacacagttaac

SEQ ID NO: 5 - DNA, 21 bp, Artificial
  Block DNA of outer forward primer for ABO gene
gtgcggccacatggagctggc

SEQ ID NO: 6 - DNA, 20 bp, Artificial
  Block DNA of outer reverse primer for ABO gene
gccgagggtagagacccagg

SEQ ID NO: 7 - DNA, 21 bp, Artificial
  Outer Forward Primer for ALDH2 gene
caaattacagggtcaactgct

SEQ ID NO: 8 - DNA, 17 bp, Artificial
  Outer and Inner Reverse Primer for ALDH2 gene
ggcaggtcctgaacctc

SEQ ID NO: 9 - DNA, 21 bp, Artificial
  Inner Forward Primer for ALDH2 gene
gtacgggctgcaggcatacac

SEQ ID NO: 10 - DNA, 21 bp, Artificial
  Block DNA of outer forward primer for ALDH2 gene
agcagttgaccctgtaatttg

SEQ ID NO: 11 - DNA, 26 bp, Artificial
  Outer Forward Primer for dystrophin gene
gatggcaaaagtgttgagaaaaagtc

SEQ ID NO: 12 - DNA, 25 bp, Artificial
  Outer Reverse Primer for dystrophin gene
ttctaccacatcccattttcttcca

SEQ ID NO: 13 - DNA, 24 bp, Artificial
  Inner Forward Primer for dystrophin gene
aggcttgaaagggcaagtagaagt

SEQ ID NO: 14 - DNA, 21 bp, Artificial
  Inner Reverse Primer for dystrophin gene
gctgatctgctggcatcttgc

SEQ ID NO: 15 - DNA, 26 bp, Artificial
  Block DNA of outer forward primer for dystrophin gene
gactttttctcaacacttttgccatc

SEQ ID NO: 16 - DNA, 25 bp, Artificial
  Block DNA of outer reverse primer for dystrophin gene
tggaagaaaatgggatgtggtagaa